(19) United States
(12) Patent Application Publication
(10) Pub. No.: US 2010/0086572 A1
(43) Pub. Date: Apr. 8, 2010

(54) RATIONAL PPI DOSAGE FORMS

(76) Inventor: RAJNEESH TANEJA, Libertyville, IL (US)

Correspondence Address: Lisa Mueller;c/o Polsinelli Shugart PC, 161 N. Clark Street, Suite 4200, Chicago, IL, 60601, US

(21) Appl. No.: 12/570,117

(22) Filed: Sep. 30, 2009

Related U.S. Application Data
(63) Continuation of application No. 11/433,288, filed on May 12, 2006, now abandoned.
(60) Provisional application No. 60/680,745, filed on May 13, 2005.
(60) Provisional application No. 60/702,206, filed on Jul. 25, 2005.

(51) Int. Cl. A61K 9/00; A61K 31/435; A61P 1/04

(52) U.S. Cl. 424/400; 514/338

(57) ABSTRACT

The invention herein provides for a continuous release dosage form (which is referred to as “dosage forms”) comprising a continuous release dosage form, which releases PPI in a first release portion directly to the gastric mucosa and a second release portion to provide for sustained plasma levels resulting in increased therapeutic efficacy.

### CROSS REFERENCE TO RELATED APPLICATIONS

The present application claims priority to U.S. Application No. 60/680,745 filed on May 13, 2005 and U.S. Application No. 60/702,206 filed on Jul. 25, 2005, both of which are herein incorporated by reference.

### BACKGROUND OF THE INVENTION

The stomach is an organ of digestion. It has a saclike shape and is located between the esophagus and the intestines. Almost every animal has a stomach.

The human stomach is a muscular, elastic, pear-shaped bag, lying crosswise in the abdominal cavity beneath the diaphragm. It changes size and shape according to is position of the body and the amount of food inside. The wall of the stomach is lined with millions of gastric glands, which together secrete 400-800 ml of gastric juice at each meal. Three kinds of cells are found in the gastric glands. These cells are parietal cells, “chief” cells and mucus-secreting cells. Parietal cells contain an enzyme known as H⁺/K⁺ adenosine triphosphate. H^{+/K}⁺ adenosine triphosphate is also referred to as an “acid pump” or “proton pump”. This transmembrane protein secretes H⁺ ions (protons) by active transport, using the energy of ATP. The concentration of H⁺ in the gastric juice can be as high as 0.15 M, giving gastric juice a pH less than 1.

Proton pump inhibitors (or “PPIs”) are a class of pharmaceutical compounds that inhibit gastric acid secretions by inhibiting H⁺/K⁺ adenosine triphosphate. It is known in the art that proton pumps can exist in either an active state or a dormant state. PPIs only bind to the active proton pumps. Examples of PPI's that are known include, but are not limited to, lansoprazole, omeprazole, and pantoprazole. PPIs are metabolized in the parietal cells to active sulfonamide metabolites that inactivate the sulthydryl group of the proton pump, therby reducing the hydrogen ion secretion (Langtry and Wilde, “An update of its pharmacological properties and clinical efficacy in the management of acid-related disorders,” Drugs, 54(3): 473-500 (1997)).

PPIs rapidly degrade in acidic environments. Therefore, dosage forms containing PPIs are generally designed to protect the PPI from the acidic environment of the stomach. Specifically, these dosage forms are designed such that a single dose of the PPI is released in the upper small intestine where the PPI can be absorbed.

With PPIs, a therapeutic response is elicited only once the plasma concentration of the drug exceeds 100 ng/mL. The maximum therapeutic response is achieved at plasma concentrations greater than 250 ng/mL. Ideally, plasma concentrations about 600 ng/mL are most desirable (namely, the therapeutic response reaches a plateau at this level). In fact, the peak plasma concentrations for PPIs typically occur within 1-3 hours after ingestion. The half-life of PPIs is generally short, usually less than 2 hours. Notwithstanding the relatively quick peak plasma levels and half-lives associated with PPIs, a prolonged therapeutic effect is attained regardless of their relatively short pharmacokinetic half life. In fact, the therapeutic effect of PPIs does not directly correlate with serum concentrations of these drugs. Accordingly, patients on PPI therapy are generally only required to take a single dosage form containing a daily dose of a PPI, usually prior to breakfast. Unfortunately, although the therapeutic effect of these drugs is longer than would otherwise be anticipated, some patients on PPI therapy experience a nighttime (nocturnal) break through event where the secretory activity of the proton pumps return. As a result, the acidity in the stomach increases and the discomfort associated with the increased acid returns.

While there have been several attempts to resolve the problem of breakthrough events, no successful remedies have yet been achieved. For example, U.S. Pat. Nos. 6,610,323, 6,274,173, and U.S. Patent Application No. 2001/0008900 relate to controlled or extended release dosage forms containing a PPI. Unfortunately, it does not appear these dosage forms are acceptable for a commercial standpoint as no controlled or extended release forms of PPIs that alleviate the nighttime breakthrough phenomenon are currently available.

The PPI, lansoprazole, is a substituted benzimidazole and is a lipophilic weak base with poor aqueous solubility at low pH. It is unstable in low pH solutions and undergoes rapid acid-catalyzed degradation. It is relatively stable at neutral or high pH. About 49%, 10%, 2% and 1% drug degrading occurs in an aqueous solution with a pH of 5, 6, 7 and 8 at 37° C., over a period of 0.5 hours respectively. Therefore, it is essential that the pH of an aqueous environment where lansoprazole resides be maintained at a neutral or alkaline pH.

The pharmacokinetic parameters and the effect of lansoprazole on gastric pH elevation have been studied by numerous investigators. In one study, Blum et al. (Blum et al., “Dose-response relationships of lansoprazole to gastric acid antisecretory effects,” Aliment. Pharmacol. Ther., 12: 321-327 (1998)) investigated the effect of varying dose regimens of lansoprazole on gastric acid suppression as measured by 24 hour intragastric pH. Lansoprazole administered as 30 mg once a day, 60 mg once a day, 60 mg twice a day and 60 mg three times a day yielded an area under the plasma concentration curve (hereinafter “AUC”) values in ng-hr/mL of 1934, 4743, 8508, and 14083 respectively.

Acid labile drugs for oral administration can be protected from gastric acidity by use of an enteric-coating. Enteric coatings are by far the most popular method of protecting an acid labile drug from gastric degradation. In this method, either the drug particles or the dosage form is coated with a polymer that does not dissolve in the low pH gastric environment, but dissolves in the alkaline environment of the small intestine. It is well understood that the drug from an enteric coated dosage form will only be released in the latter part of the duodenum or further down in the gastrointestinal tract where the pH exceeds 5.5. Currently, lansoprazole is administered in the form of enteric-coated granules filled in a hard gelatin capsule. (Delhotal et al., “Clinical pharmacokinetics of lansoprazole,” Clin. Pharmacokinet., 28(6): 458-70 (1995)). This enteric coat dissolves at a pH greater than 5.5.

There is a need in the art for a dosage form containing a PPI that can reliably provide a full day of therapeutic effect, including the alleviation of the nighttime breakthrough phenomenon, while being administered on a once a day basis.

### SUMMARY OF THE INVENTION

The present invention is directed to a continuous release dosage form of a proton pump inhibitor (PPI). The PPI is released from the dosage form in two portions, namely, a first release portion and a second release portion. The first release portion is released into the gastric mucosa such that the PPI acts directly at the local site of action in the gastric mucosa. The second release portion is released to provide a sustained plasma concentration.

In another embodiment, in a fasted state, at least 10% of the PPI in the dosage form is released in the first release portion and the remaining PPI is released in the second release portion over a period of at least four hours.

In another embodiment, the PPI in the dosage form is lansoprazole.

In a preferred embodiment, the lansoprazole in the dosage form comprises a dosage range of from between 60-240 milligrams (mg.) of lansoprazole. As used herein, the term “dosage range” refers to the amount of the PPI, in this case lansoprazole, present in the dosage form.

In a more preferred embodiment, the lansoprazole in dosage form comprises a dosage range of from between 90-180 mg. of lansoprazole.

In the most preferred embodiment, the lansoprazole in the dosage form comprises approximately 120 mg. of lansoprazole.

In yet another embodiment, the invention is directed to a method of treating a gastrointestinal disorder comprising administering to a patient in need thereof a continuous release dosage form as described above.

In yet another embodiment, the present invention also relates to a non-enteric coated dosage form for immediate and sustained delivery of at least one PPI to parietal cells in the gastric mucosa in a patient in need of treatment thereof. Such a dosage form comprises at least one PPI and at least one non-enteric coating and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/mL for at least 5 hours after ingestion of said dosage form by said patient. Optionally, said dosage form may also comprise at least one stabilizer.

In another embodiment, the PPI in the dosage form is lansoprazole.

In a preferred embodiment, the lansoprazole in the dosage form comprises a dosage range of from between 60-240 milligrams (mg.) of lansoprazole. As used herein, the term “dosage range” refers to the amount of the PPI, in this case lansoprazole, present in the dosage form.

In a more preferred embodiment, the lansoprazole in dosage form comprises a dosage range of from between 90-180 mg. of lansoprazole.

In the most preferred embodiment, the lansoprazole in the dosage form comprises approximately 120 mg. of lansoprazole.

In yet another embodiment, the invention is directed to a method of treating a gastrointestinal disorder comprising administering to a patient in need thereof a continuous release dosage form as described above.

In yet another embodiment, the present invention also relates to a non-enteric coated dosage form for immediate and sustained delivery of at least one PPI to parietal cells in the gastric mucosa in a patient in need of treatment thereof. Such a dosage form comprises at least one PPI and at least one non-enteric coating and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/ml, for at least 5 hours after ingestion of said dosage form by said patient and further wherein said dosage form is a dosage form other than a micropump dosage form. Optionally, said dosage form may also comprise at least one stabilizer.

In another embodiment, the PPI in the dosage form is lansoprazole.

In a preferred embodiment, the lansoprazole in the dosage form comprises a dosage range of from between 60-240 milligrams (mg.) of lansoprazole. As used herein, the term “dosage range” refers to the amount of the PPI, in this case lansoprazole, present in the dosage form.

In a more preferred embodiment, the lansoprazole in dosage form comprises a dosage range of from between 90-180 mg. of lansoprazole.

In the most preferred embodiment, the lansoprazole in the dosage form comprises approximately 120 mg. of lansoprazole.

In yet another embodiment, the invention is directed to a method of treating a gastrointestinal disorder comprising administering to a patient in need thereof a continuous release dosage form as described above.

In yet another embodiment, the present invention is directed to a method of treating chronic cough in a patient suffering from acid reflux. The method involves the step of administering to said patient at least one of the hereinbefore described dosage forms of the present invention.

In yet another embodiment, the present invention is directed to a method of treating a patient suffering from ulcerative colitis. The method involves the step of administering to said patient at least one of the hereinbefore described dosage forms of the present invention.

### DESCRIPTION OF THE FIGURES

FIG. 1 is a graph showing the mean 24-hour gastric pH on day 5 after administering the different medium-release and fast-release dosage forms of the present invention containing lansoprazole.

FIG. 2 is a graph showing a comparison on day 5 of the 24 hour pH profile of lansoprazole 60 mg versus esomeprazole 40 mg.

FIG. 3 is a graph showing a comparison on day 5 of the 24 hour pH profile of Lansoprazole SR (Example 1) versus esomeprazole 40 mg.

FIG. 4 is a schematic representation of a Lansoprazole SR microparticle.

### DETAILED DESCRIPTION OF THE INVENTION

### Definitions

As used in this specification and the appended claims, the singular forms “a,” “an” and “the” include plural references unless the context clearly dictates otherwise. Thus, for example, reference to “an active agent” includes a single active agent as well two or more different active agents in combination.

In describing and claiming the present invention, the following terminology will be used in accordance with the definitions set out below.

As used herein, the term “chronic cough” refers to a cough that last for a period of at least one (1) week, preferably at least two (2) weeks and most preferably at least three (3) weeks. Methods of treating chronic cough using PPIs are disclosed in Chung, Clint. Exp. Allergy, 35:245-246 (2005).

The term “dosage form” refers to any solid object, semi-solid, or liquid composition designed to contain a specific pre-determined amount (i.e. dose) of a certain PPI. Suitable dosage forms may be pharmaceutical drug delivery systems, including those for oral administration, buccal administration, rectal administration, topical or mucosal delivery or subcutaneous implants, or other implanted drug delivery systems and the like. Preferably, the dosage forms of the present invention are considered to be solid, however, they may containing liquid or semi-solid components. More preferably, the dosage form is an orally administered system for delivering a PPI to a patient. The dosage form of the present invention exhibits continuous release of the PPI.

By an “effective amount” or a “therapeutically effective amount” of an PPI is meant a nontoxic but sufficient amount of the PPI to provide the desired effect. The amount of PPI that is “effective” will vary from subject to subject, depending on the age and general condition of the individual, the particular PPI or PPIs, and the like. Thus, it is not always possible to specify an exact “effective amount.” However, an appropriate “effective amount” in any individual case may be determined by one of ordinary skill in the art using routine experimentation.

As used herein, the terms “fasted” patient or “fasting conditions” refers to administration of a dosage form in the morning 30 to 60 minutes prior to consumption of breakfast by a patient.

As used herein, the term “gastric mucosa” refers to the lining of the stomach. This lining is composed of simple columnar epithelium cells that are perforated by numerous small holes called foveolae gastricae. The foveolae are the openings of epithelial invaginations, the gastric pits, which penetrate the lamina propria to various depths. The pits serve as ducts for the branched tubular gastric glands, Each gland has 3 regions: an isthmus at the bottom of the pit, a straight neck that penetrates deeper into the lamina propria (perpendicular to the surface), and a coiled base that penetrates deeper still and ends blindly just above the muscularis mucosae. The gastric mucosa comprises parietal cells, “chief” cells, mucus-secreting cells as well as other epithelial cell types.

As used herein, the term “gastrointestinal disorder” refers to any disease or disorder of the upper and lower gastrointestinal tract of a patient including, for example, inflammatory bowel disease, Crohn's disease, irritable bowel syndrome, ulcerative colitis, peptic ulcers, stress ulcers, bleeding peptic ulcers, duodenal ulcers, infectious enteritis, colitis, diverticulitis, gastric hyperacidity, dyspepsia, gastroparesis, Zollinger-Ellison syndrome, gastroesophageal reflux disease (i.e., acid reflux), Helicobacter Pylori associated disease, short-bowel (anastomosis) syndrome, hypersecretory states associated with systemic mastocytosis or basophilic leukemia and hyperhistaminemia that result, for example, from neurosurgery, head injury, severe body trauma or burns.

As used herein, the term “lower gastrointestinal tract” refers to the ileum, the colon, the cecum and the rectum.

As used herein, the term “micropump dosage form” refers to microcapsules of the reservoir kind disclosed in U.S. Pat. No. 6,022,562. More specifically, U.S. Pat. No. 6,022,562 discloses microcapsules of reservoir kind containing at least one medicinal and nutritional active principle (AP), with the exclusion of acetylsalicylic acid (ASA), which are intended for oral administration, characterized:

in that they consist of particles of AP each coated with at least one coating film of the following specific composition:

1—at least one film-forming polymer (P1) which is insoluble in the liquids of the digestive tract, present in a quantity of 50 to 90%, preferably 50 to 80% by weight of dry matter of the whole coating composition, and consisting of at least one non-hydrosoluble cellulose derivate, ethylcellulose and/or cellulose acetate being preferred;

2—at least one nitrogen-containing polymer (P2), present in a quantity of 2 to 25, preferably 5 to 15% by weight of dry matter of the whole coating composition, and consisting of at least one polyacrylamide and/or one poly-N-vinylamide and/or one poly-N-vinyl-lactame, the polyacrylamide and/or the polyvinylpyrrolidone being preferred;

3—at least one plasticizer present in a quantity of 2 to 20%, preferably 4 to 15% by weight of dry matter of the whole coating composition, and consisting of at least one of the following compounds: glycerol esters, phtalates, citrates, sebacates, cetylalcohol esters, castor oil and cutin, castor oil being particularly preferred;

4—at least one surface-active and/or lubricating agent, present in a quantity of 2 to 20%, preferably 4 to 15% by weight of dry matter of the whole coating composition, and chosen from anionic surfactants, preferably the alkali metal or alkakine-earth metal salts of fatty acids, stearic acid and/or oleic acid being preferred, and/or from nonionic surfactants, preferably polyoxyethylenated esters of sorbitan and/or polyoxyethylenated esters of sorbitan and/or polyoxyethylenated derivatives of castor oil, and/or from lubricants such as stearates, preferably calcium, magnesium, aluminium or zinc stearate, or such as stearylfumarate, preferably sodium stearylfimarate, and/or glyceryl behenate, said agent comprising only one or a mixture of the above products;

in that they have a particle size of between 50 and 1000 microns, preferably of between 100 and 750 microns and, more preferably, of between 100 and 500 microns;

in that they are designed so as to be able to remain in the small intestine for a period of at least about 5 hours, preferably of at least about 7 hours and, even more preferably, for a period of between about 8 hours and about 24 hours, and permitting so the absorption of the AP during at least part of their residence in the small intestine.

The term “patient” refers to an animal, preferably a mammal, including a human or non-human. The terms patient and subject may be used interchangeably herein.

By “pharmaceutically acceptable,” such as in the recitation of a “pharmaceutically acceptable excipient,” or a “pharmaceutically acceptable additive,” is meant a material that is not biologically or otherwise undesirable, i.e., the material may be incorporated into a pharmaceutical composition administered to a patient without causing any undesirable biological effects.

As used herein, the term “protein pump inhibitor(s)” or “PPI” refers to compounds that inhibit the hydrogen-potassium (H⁺/K⁺) adenosine triphosphate enzyme system. Many PPIs are commercially available and are commonly prescribed for gastroesophageal reflux disease (GERD) as well as other indications. Any compound having PPI activity can be used in the present dosage forms and examples of such PPI's include, but are not limited to, lansoprazole, ilaprazole, omeprazole, rabeprazole, and pantoprazole, as well as, for example, prodrugs and enantiomers of any of the above. The term “PPI” and “drug” are used interchangeably herein.

As used herein, the term “stabilizer” refers to any chemical, compound or material that minimizes the degradation of the PPI by the acidic environment of the stomach. Examples of stabilizers include Group IA or Group IIA salts (such as but not limited to, sodium salts, calcium salts, magnesium salts, etc.), polymers, sodium alginate, sterols, fatty alcohols, etc.

Examples of polymers that can be used include, but are not limited to, semipermeable homopolymers, semipermeable copolymers, and the like. Preferably, the polymers cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like.

Examples of semipermeable polymers include a member selected from the group consisting of cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%; cellulose diacetate having a DS of 1 to 2 and an acetyl content of 21 to 35%; cellulose triacetate having a DS of 2 to 3 and an acetyl content of 34 to 44.8%; and the like. More specific cellulosic polymers include cellulose propionate having a DS of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a DS of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a DS of 2.6 to 3, such as cellulose trivalerate, cellulose trilamate, cellulose tripalmitate, cellulose trioctanote and cellulose tripropionate; cellulose diesters having a DS of 2.2 to 2.6, such as cellulose disuccinate, cellulose dipalmitate, cellulose dioctanoate, cellulose dicarpylate, and the like; and mixed cellulose esters, such as cellulose acetate valerate, cellulose acetate succinate, cellulose propionate succinate, cellulose acetate octanoate, cellulose valerate palmitate, cellulose acetate heptonate, and the like. Semipermeable polymers are known in U.S. Pat. No. 4,077,407, and they can be synthesized by procedures described in Encyclopedia of Polymer Science and Technology, Vol. 3, pp. 325-354 (1964), Interscience Publishers Inc., New York, N.Y.

Semi-permeable polymers comprise cellulose acetaldehyde dimethyl acetate; cellulose acetate ethylcarbamate; cellulose acetate methyl carbamate; cellulose dimethylaminoacetate; semipermeable polyamide; semipermeable polyurethanes; semipermeable sulfonated polystyrenes; cross-linked selectively semipermeable polymers formed by the coprecipitation of an anion and a cation, as disclosed in U.S. Pat. Nos. 3,173,876; 3,276,586; 3,541,005; 3,541,006 and 3,546,142; semipermeable polymers, as disclosed by Loeb, et al. in U.S. Pat. No. 3,133,132; semipermeable polystyrene derivatives; semipermeable poly(sodium styrenesulfonate); semipermeable poly(vinylbenzyltremethylammonium chloride); and semipermeable polymers exhibiting a fluid permeability of 10⁻⁵ to 10⁻² (cc. mil/cm hr.atm), expressed as per atmosphere of hydrostatic or osmotic pressure differences across a semipermeable wall. The polymers are known to the art in U.S. Pat. Nos. 3,845,770; 3,916,899 and 4,160,020; and in Handbook of Common Polymers, Scott and Roff (1971) CRC Press, Cleveland, Ohio.

Examples of sterols that can be used are phytosterols (such as ergosterols, stigmasterol, sitosterol, brassicasterol and campesterol) or zoosterols (such as cholesterol and lanosterol).

The fatty alcohols that can be used can be linear, saturated or unsaturated primary alcohols having 10-30 carbon atoms. Examples of fatty alcohols that can be used include, but are not limited to cetyl alcohol, myristyl alcohol or stearyl alcohol.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

As used herein, the term “ulcers” refers to lesions of the upper gastrointestinal tract lining that are characterized by loss of tissue. Such ulcers include gastric ulcers, duodenal ulcers and gastritis.

As used herein, the term “upper gastrointestinal tract” refers to the esophagus, the stomach, the duodenum and the jejunum.

### DESCRIPTION OF THE INVENTION

The present invention relates to continuous release dosage forms comprising at least one drug. More specifically, the dosage forms of the present invention have been designed so that the PPI is released from the dosage form in two portions, namely, a first release portion and a second release portion.

The first release portion of the dosage form of the present invention immediately releases at least 10% of the drug or greater than 3 mg of drug in the upper gastrointestinal tract, preferably, in the gastric mucosa, within thirty to ninety minutes after ingestion of the dosage form by a patient under fasting conditions. The immediate release of drug directly into the upper gastrointestinal tract, preferably into the gastric mucosa of the stomach, occurs where the acid producing parietal cells reside. More specifically, the release of the drug (i.e, the PPI) in the gastric mucosa, results in the drug being present directly at the site of action, namely at the proton pumps (i.e the H⁺/K⁺-adenosine triphosphate enzyme), without the drug first having to pass through the systemic circulation prior to delivery to the parietal cell. In other words, the immediate release of the drug in the gastric mucosa allows for a higher concentration of drug to be present at the proton pumps without the drug first having to pass through the systemic circulation. This results in a quicker onset of action with respect to inhibiting the active proton pumps.

After the immediate release of the first release portion of the drug into the upper gastrointestinal tract, the second release portion of the drug occurs. Specifically, the remaining drug, during the second release portion, is released continuously from the dosage form over an at least four (4) hour period after ingestion. The release of the remaining portion of the drug over the at least four hour period results in a sustained plasma concentration of the drug in the patient. More specifically, a sustained plasma concentration in an amount of greater than 300 ng/mL of PPI is achieved for at least 5 hours after ingestion of said dosage form by said patient. Preferably, the plasma concentration is sustained in an amount of greater than 300 ng/mL for a period of at least 8 hours.

The dosage form of the present invention can optionally contain at least one stabilizer. The amount of PPI to stabilizer present in the dosage form is at least equimolar. For example: a 1:1 ratio of PPI to stabilizer, 2:2 ratio of PPI to stabilizer, etc.

Unlike most of the other commercially available PPI dosage forms, the release of the drug from the dosage forms of the present invention does not require or depend upon the use of an enteric coating. Instead, the dosage forms of the present invention may not contain any coating whatsoever or can employ a non-enteric coating. Non-enteric coatings are well known in the art and include, but are not limited to, which are well known in the art. Examples of non-enteric coatings that can be used include, but are not limited to, ethylcelluloses (such as Surelease®), poly(ethylacrylate-methylmethacrylates) (such as Eudragit® NE30D), and poly(ethylacrylate-methylmethacrylate) triethyl animonioethyl methacrylate chlorides) (such as Eudragit® RL30D). The amount of ethylcellulose that can be used can range from 3-25% by weight. The amount of poly(ethylacrylate-methylmethacrylate) that can be used can range from 20-50% by weight. The amount of poly(ethylacrylate-methylmethacrylate) triethyl ammonioethyl methacrylate chlorides) that can be used can range from 20-40% by weight.

More specifically, the dosage forms of the present invention, unlike enteric coated PPI containing dosage forms, do not have to be transported to the alkaline environment in the small intestine before the drug is released. With enteric coated PPI containing dosage forms, after such dosage forms are administered orally, the PPI is absorbed into the bloodstream and then presented to the parietal cells via systemic circulation. Once the PPI reaches the parietal cells, the PPI binds to the proton pumps and inhibits acid production. With enteric coated PPI containing dosage forms, there is never any direct, physical contact between the PPI and the stomach wall and thus the parietal cells. In contrast, the dosage forms of the present invention not only release the PPI earlier than enteric coated dosage forms, but also, place the PPI in direct physical contact with the stomach wall and hence the parietal cells. While not wishing to be bound by any theory, the inventors of the present invention believe that when the drug is placed in direct physical contact with the stomach wall that part of the drug is directly absorbed across the gastric epithelium into the parietal cells, thus providing a high local concentration of the PPI at the site of drug action. The inventors believe that this combination of local and systemic drug exposure to the parietal cells should provide higher drug concentrations in the parietal cells compared to enteric coated PPI containing dosage forms.

Non-enteric coated PPI dosage forms are known in the art (See, Sharma, V. K., “Comparison of 24-hour intragastric pH using four liquid formulations of lansoprazole and omeprazole,” Am J Health-Syst Pharm. 56: S18-21 (1999) and Phillips et al., “A prospective study of simplified omeprazole suspension for the prophylaxis of stress-related mucosal damage,” Crit. Care Med. 24(11): 1793-1800 (1996)). However, in both Sharma and in Phillips et al., the enteric-coated PPI dosage form is mixed with sodium bicarbonate solution prior to administration to the patient. This alkaline solution (namely, the sodium bicarbonate solution) dissolves the enteric coating of the dosage form and the drug becomes suspended in this solution. Non-enteric coated dosage forms containing PPIs are commercially available (For example, Zegrie). However, these dosage forms do not provide extended, continuous release of the PPI.

Based upon the description provided herein, the dosage forms of the present invention can be used as part of a rationalized approach for delivering at least one PPI to a patient in need of treatment thereof. The dosage forms of the present invention minimize a patient's exposure to the PPI while also maintaining a sustained period of acid suppression that is superior to currently available commercial products.

Additionally, the dosage forms of the present invention are expected to provide superior night-time acid control as a result of the sustained plasma concentrations of the PPI as described herein. It is believe that such sustained plasma concentrations will benefit patients suffering from nocturnal acid breakthrough.

The dosage forms of the present invention provide a sustained plasma drug concentration of PPI that is sufficient to effectively block the proton pumps in the gastric mucosa for long periods of time when compared to dosage forms known in the art. More specifically, the dosage forms of the present invention maintain a stomach pH greater than 3 for a period of at least 20 hours. Preferably, the dosage forms of the present invention maintain a stomach pH greater than 4 for a period of at least 12 hours. Therefore, the dosage forms of the present invention can be used to treat patients suffering from one or more gastrointestinal disorders and upper respiratory tract inflammation (such as chronic cough).

Proton pumps have been recently identified in larynx of humans, particularly the serous cells and ducts of submucosal glands (See Altman, K., et al., Laryngoscope, 113(11):1927-1930 (November 2003)). It is known in the art that some patients suffering from GERD also experience chronic laryngitis and/or chronic cough. Therefore, the dosage forms of the present invention can be used to treat patients suffering from GERD who also experience chronic laryngitis and/or chronic cough by also effectively blocking the activity of the proton pumps in the larynx.

Ulcerative colitis, which is also called colitis or proctitis, is a disease that causes inflammation and sores, which are referred to as “ulcers”, in the lining of the large intestine. The inflammation usually occurs in the rectum and lower part of the colon, but it can affect the entire colon. Ulcerative colitis rarely affects the small intestine except at the end section, which is referred to as the terminal ileum. Two types of proton pumps have been identified in the mucosa of the distal rabbit colon. One of these pumps was shown to have similar characteristics to the gastric proton potassium exchanger (Kaunitz, J. D. et al., J Biol. Chem., 261:14005-14010 (1986)). It has been demonstrated in the art that lansoprazole inhibits the formation of experimentally induced-induced colitis. Currently available formulations of PPI are not effective in treating ulcerative colitis because the PPI contained in these formulations are rapidly absorbed in the small intestine. In contrast, the dosage forms of the present invention can be used to deliver at least a portion of the PPI to the large intestine, specifically at the site of inflammation in patients suffering from ulcerative colitis.

Additionally, the dosage forms of the present invention can be used to treat solid tumors in patients suffering from such tumors. More specifically, the dosage forms of the present invention can be used in combination with chemotherapy and other tumor treatments to reduce solid tumor size and mass.

The benefits of this invention are not limited to a particular type of dosage form having a specific mechanism of drug release. The enhanced efficacy, especially in alleviating nocturnal breakthrough events can be obtained with any dosage form suitable for releasing a PPI such that, for example, a continuous release of the drug meets the drug delivery criteria mentioned above. In view of the discovery of the drug delivery criteria, the method of delivery of the PPI is a matter of choice for those skilled in the art.

Many types of continuous drug release dosage forms are known in the art. For example, controlled or extended release, and pulsed release dosage forms are known. Any type of continuous drug release dosage form can be used in the present invention, including matrix systems, osmotic pumps, and membrane controlled systems (also referred to as reservoir systems). Each of these systems is described in greater detail below. A detailed discussion of such dosage forms may also be found in: (i) Handbook of pharmaceutical controlled release technology, ed. D. L. Wise, Marcel Dekker, Inc. New York, N.Y. (2000), and (ii) and Treatise on controlled drug delivery, fundamentals, optimization, and applications, ed. A. Kydonieus, Marcel Dekker, Inc. New York, N.Y. (1992).

Matrix systems are well known in the art. In a matrix system, the drug is homogenously dispersed in a polymer and optionally, conventional excipients. This so-called admixture is typically compressed under pressure to produce a tablet. Drug is released from this tablet by diffusion and erosion. Matrix systems typically employ a pharmaceutically acceptable polymer such as a water-soluble hydrophilic polymer, or a water insoluble hydrophobic polymer (including waxes). Examples of suitable water soluble polymers include polyvinylpyrrolidine, hydroxypropylcellulose, hydroxypropylmethyl cellulose, methyl cellulose, vinyl acetate copolymers, polysaccharides (such as alignate, xanthum gum, etc.), polyethylene oxide, methacrylic acid copolymers, maleic anhydride/methyl vinyl ether copolymers and derivatives and mixtures thereof. Examples of suitable water insoluble polymers include acrylates, cellulose derivatives such ethylcellulose or cellulose acetate, polyethylene, methacrylates, acrylic acid copolymers and high molecular weight polyvinylalcohols. Examples of suitable waxes include fatty acids and glycerides.

The dosage forms of the present invention also typically includes pharmaceutically acceptable excipients. As is well known to those skilled in the art, pharmaceutical excipients are routinely incorporated into solid dosage forms. This typically is done to ease the manufacturing process as well as to improve the performance of the dosage form. Common excipients include diluents or bulking agents, lubricants, binders, etc.

Diluents, or fillers, can be added to, for example, increase the mass of an individual dose to a size suitable for tablet compression. Suitable diluents include, for example, powdered sugar, calcium phosphate, calcium sulfate, microcrystalline cellulose, lactose, mannitol, kaolin, sodium chloride, dry starch, and sorbitol.

Lubricants can be incorporated into a dosage form for a variety of reasons. They reduce friction between the granulation and die wall during compression and ejection. This prevents, for example, a granulate from sticking to the tablet punches, and facilitates its ejection from the tablet punches. Examples of suitable lubricants include talc, stearic acid, vegetable oil, calcium stearate, zinc stearate, and magnesium stearate.

Glidant's can also be incorporated into a dosage form, typically for purposes of improving the flow characteristics of the granulation. Examples of suitable glidant's include talc, silicon dioxide, and cornstarch.

Binders also may be incorporated into the dosage form. Binders are typically utilized if the manufacture of the dosage form uses a granulation step. Examples of suitable binders include povidone, polyvinylpyrrolidone, xanthan gum, cellulose gums such as carboxymethylcellulose, methyl cellulose, hydroxypropylmethylcellulose, hydroxycellulose, gelatin, starch, and pregelatinized starch.

Other excipients that may be incorporated into the dosage form include preservatives, antioxidants, or any other pharmaceutically acceptable excipient commonly used in the pharmaceutical industry.

The amount of excipients used in the dosage form will correspond to that typically used in a matrix system. The total amount of excipients, fillers and extenders, and the like typically will vary from about 10% to about 80% by weight of the dosage form.

Matrix dosage forms are generally prepared using standard techniques well known in the art. Typically, they are prepared by dry blending the polymer, filler, drug, and other excipients followed by granulating the mixture using an alcohol until proper granulation is obtained. The granulation is done by methods known in the art. The wet granules are dried in a fluid bed dryer, sifted and ground to appropriate size. Lubricating agents are mixed with the dried granulation to obtain the final dosage form.

In an osmotic pump system, a tablet core is encased by a semipermeable membrane having at least one orifice. The semipermeable membrane is permeable to water, but impermeable to the drug. When the system is exposed to body fluids, water will penetrate through the semipermeable membrane into the tablet core containing osmotic excipients and the active drug. Osmotic pressure increases within the dosage form and drug is released through the orifice in an attempt to equalize pressure.

In more complex pumps, the tablet core contains multiple internal compartments. For example, the first compartment may contain the drug and the second compartment may contain a polymer that swells on contact with fluid. After ingestion, this polymer swells into the drug containing compartment at a predetermined rate and forces drug from the dosage form at that rate. Such dosage forms are often used when are zero order release profile is desired.

Osmotic pumps are well known in the art and have been described in the literature. U.S. Pat. Nos. 4,088,864; 4,200,098; and 5,573,776; all of which are hereby incorporated by reference, describe osmotic pumps and methods for their manufacture. Osmotic pumps containing compounds, such as omeprazole, have been described in U.S. Pat. No. 5,178,867, the contents of which are hereby incorporated by reference.

As a general guideline, osmotic pumps are typically formed by compressing a tablet of an osmotically active drug (or an osmotically inactive drug in combination with an osmotically active agent or osmagent) and then coating the tablet with a semipermeable membrane that is permeable to an exterior aqueous-based fluid but impermeable to the passage of drug and/or osmagent. One or more delivery orifices may be drilled through the semipermeable membrane wall. Alternatively, orifice(s) through the wall may be formed in situ by incorporating leachable pore forming materials in the wall. In operation, the exterior aqueous based fluid is imbibed through the semipermeable membrane wall and contacts the drug and/or salt to form a solution or suspension of the drug. The drug solution or suspension is then pumped out through the orifice as fresh fluid is imbibed through the semipermeable membrane.

As previously mentioned, osmotic pumps may contain multiple distinct compartments. The first compartment may contain the drug as described above, and the second compartment may contain an expandable driving member consisting of a layer of a swellable hydrophilic polymer, which operates to diminish the volume occupied by the drug, thereby delivering the drug from the device at a controlled rate over an extended period of time. Alternatively, the compartments may contain separate doses of the drug.

Typical materials for the semipermeable membrane include semipermeable polymers known to the art as osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, cross-linked selectively semipermeable polymers formed by the coprecipitation of a polyanion and a polycation as disclosed in U.S. Pat. Nos. 3,173,876; 3,276,586; 3,541,005; 3,541,006; and 3,546,142, semipermeable polymers as disclosed by Loeb and Sourirajan in U.S. Pat. No. 3,133,132, lightly cross-linked polystyrene derivatives, cross-linked poly(sodium styrene sulfonate), poly(vinylbenzyltrimethyl ammonium chloride), cellulose acetate having a degree of substitution up to 1 and an acetyl content up to 50%, cellulose diacetate having a degree of substitution of 1 to 2 and an acetyl content of 21 to 35%, cellulose triacetate having a degree of substitution of 2 to 3 and an acetyl content of 35 to 44.8%, as disclosed in U.S. Pat. No. 4,160,020.

The osmotic agent present in the pump, which may be used when the drug itself is not sufficiently osmotically active, are osmotically effective compounds soluble in the fluid that enters the pump, and exhibits an osmotic pressure gradient across the semipermeable wall against the exterior fluid. Osmotically effective osmagents useful for the present purpose include magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, mixtures thereof, and the like. The osmagent is usually present in an excess amount, and it can be in any physical form, such as particle, powder, granule, and the like. The osmotic pressure in atmospheres of the osmagents suitable for the invention will be greater than zero and generally up to about 500 atm, or higher.

The expandable driving member typically is a swellable, hydrophilic polymer which interacts with water and aqueous biological fluids and swells or expands to an equilibrium state. The polymers exhibit the ability to swell in water and retain a significant portion of the imbibed water within the polymer structure. The polymers swell or expand to a very high degree, usually exhibiting a 2 to 50 fold volume increase. The polymers can be noncross-linked or cross-linked. The swellable, hydrophilic polymers are in one presently preferred embodiment lightly cross-linked, such cross-links being formed by covalent ionic bonds or hydrogen bonds. The polymers can be of plant, animal or synthetic origin. Hydrophilic polymers suitable for the present purpose include poly(hydroxy alkyl methacrylate) having a molecular weight of from 30,000 to 5,000,000; kappa carrageenan, polyvinylpyrrolidone having molecular weight of from 10,000 to 360,000; anionic and cationic hydrogels; polyelectrolyte complexes; polyvinyl alcohol) having a low acetate residual, cross-linked with glyoxal, formaldehyde, or glutaraldehyde and having a degree of polymerization from 200 to 30,000; a mixture of methyl cellulose; cross-linked agar and carboxymethyl cellulose; a water insoluble, water swellable copolymer produced by forming a dispersion of finely divided copolymer of maleic anhydride with styrene, ethylene, propylene, butylene or isobutylene cross-linked with from 0.001 to about 0.5 moles of saturated cross-linking agent per mole of maleic anhydride in copolymer; water swellable polymers of N-vinyl lactams, and the like.

The expression “orifice” as used herein comprises means and methods suitable for releasing the drug from an osmotic system. The expression includes one or more apertures or orifices which have been bored through the semipermeable membrane by mechanical procedures. Alternatively it may be formed by incorporating an erodible element, such as a gelatin plug, in the semipermeable membrane. In cases where the semipermeable membrane is sufficiently permeable to the passage of drug, the pores in the membrane may be sufficient to release the PPI in amounts sufficient to meet the plasma threshold. In such cases, the expression “passageway” refers to the pores within the membrane wall even though no bore or other orifice has been drilled there through. A detailed description of osmotic passageways and the maximum and minimum dimensions for a passageway are disclosed in U.S. Pat. Nos. 3,845,770 and 3,916,899, the disclosures of which are incorporated herein by reference.

The osmotic pumps of this invention can be manufactured by standard techniques. For example, in one embodiment, the drug and other ingredients that may be housed in one area of the compartment adjacent to the passageway, are pressed into a solid possessing dimension that corresponds to the internal dimensions of the area of the compartment the agent will occupy, or the agent and other ingredients and a solvent are mixed into a solid or semisolid form by conventional methods such as ballmilling, calendaring, stirring or rollmilling, and then pressed into a preselected shape. Next, a layer of a hydrophilic polymer is placed in contact with the layer of agent in a like manner, and the two layers surrounded with a semipermeable wall. The layering of agent formulation and hydrophilic polymer can be fabricated by conventional two-layer press techniques. The wall can be applied by molding, spraying or dipping the pressed shapes into a wall forming material. Another and presently preferred technique that can be use for applying the wall is the air suspension procedure. This procedure consists of suspending and tumbling the pressed agent and dry hydrophilic polymer in a current of air and a wall forming composition until the wall is applied to the agent-hydrophilic polymer composite. The air suspension procedure is described in U.S. Pat. No. 2,799,241; J. Am. Pharm. Assoc., Vol. 48, pp. 451-459, (1979). Other standard manufacturing procedures are described in Modern Plastics Encyclopedia, Vol. 46, pp. 62-70 (1969); and in Pharmaceutical Sciences, by Remington, Fourteenth Edition, pp. 1626-1678 (1970), published by Mack Publishing Company, Easton, Pa.

Reservoir systems also are well known in the art. This technology is also commonly referred to as microencapsulation, bead technology, or coated tablets. Small particles of the drug are encapsulated with pharmaceutically acceptable polymer. This polymer, and its relative quantity, offers a predetermined resistance to drug diffusion from the reservoir to the gastrointestinal tract. Thus drug is gradually released from the beads into the gastrointestinal tract and provides the desired sustained release of the compound.

These dosage forms are well known in the art. U.S. Pat. Nos. 5,286,497 and 5,737,320, both of which are hereby incorporated by reference, describe such dosage forms and their methods of production. U.S. Pat. Nos. 5,354,556, 4,952,402, and 4,940,588, all of which are hereby incorporated by reference, specifically discuss using such technology to produce sustained release dosage forms. As further guidance, however, a pellet is formed with a core of a drug, optionally in association with conventional excipients. This core is then coated with one, or more, pharmaceutically acceptable polymers. Often, the coating polymer is an admixture of a major proportion of a pharmaceutically acceptable water insoluble polymer and a minor proportion of a pharmaceutically acceptable water soluble polymer.

The central core may be prepared by a number of techniques known in the art. Typically the drug is bound to an inert carrier with a conventional binding agent. The inert carrier is typically a starch or sugar sphere. Before the drug is bound to the inert carrier, it is typically blended with conventional excipients to expedite its handling and to improve the properties of the final dosage form. These excipients are identical to those described above for the matrix systems. The quantity of these excipients can vary widely, but will be used in conventional amounts. The central core is then produced by utilizing a binding agent to attach the powdered drug blend to the solid carrier. This can be accomplished by means known in the art for producing pharmaceutical beads. Suitable means include utilization of a conventional coating pan, an automatic coating machine, or a rotogranulator. The production of these central cores is described in more detail in Pharmaceutical Pelletization Technology, ed. I. Ghebre-Sellassie, Marcel Dekker, Inc. New York, N.Y. (1989).

The second major component of a reservoir system is the polymeric coating. As noted above, the polymeric coating is responsible for giving the beads their release characteristics. The polymeric coating may be applied to the central core using methods and techniques known in the art. Examples of suitable coating devices include fluid bed coaters and pan coaters. The application techniques are described in more detail in: i) Aqueous polymeric coatings for pharmaceutical dosage forms, ed. J. W. McGinity, Marcel Dekker, Inc. New York, N.Y. (1997); and ii) Pharmaceutical Dosage Forms: Tablets Vol. 3. ed. H. A. Lieberman, L. Lachman and J. B. Schwartz, Marcel Dekker, Inc. New York, N.Y. pp. 77-287, (1990).

Examples of suitable polymers include ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene oxide), poly(ethylene terephthalate), poly(vinyl isobutyl ether), poly(vinyl acetate), poly(vinyl chloride) or polyurethane or mixtures thereof.

Once the beads have been prepared, they may be filled into capsules as is known in the art. Alternately, they may be pressed into tablets using techniques conventional in the art.

Pulsed release systems, the other broad category of modified release dosage forms, are also well known in the art. Pulsed release systems generally involve a first drug release and a second drug release separated by a predetermined period of time or site of release. Pulsed release systems also may include a combination of immediate release and extended release. Multiple formulation configurations are suitable for pulsed release dosage forms.

For example, osmotic pumps also are suitable for purposes of pulsatile drug release and have been described in U.S. Pat. Nos. 5,017,381 and 5,011,692, both of which are herein incorporated by reference. Generally, the osmotic pump containing the drug is formed and then overcoated with a layer of a drug to provide for two releases of the drug, one from the coating layer and another from the osmotic pump.

Particle or granule systems have also been proposed for purposes of providing a pulsed release of drug. U.S. Pat. No. 6,228,398 (incorporated herein by reference) teach the use of such systems for a pulsed release of a drug. Such systems typically use distinct populations of drug containing particles to achieve a pulsed release. The populations employ different coating polymers, such as those mentioned above, to release the drug at different points in time or location. For example, polymers having different dissolution pHs are commonly used for this purpose. Hence, one population of granules can be coated with a polymer that begins dissolving at a pH of 6 and another population of granules can be coated with a polymer that begins dissolving at a pH of 6.5 to achieve a pulsed release. In this manner, the first population of granules would release the drug in the upper small intestine while the second population of the granules would release the drug further down stream and therefore at a later time.

It will be understood, of course, that any of the dosage forms used in accordance with the present invention may employ an enteric coating or buffering systems such as those described in U.S. Pat. Nos. 6,849,346; 5,026,560; 5,045,321; 4,786,505; and 6,849,346 (all of which are herein incorporated by reference) for purposes of protecting the PPI.

The dosage forms of the present invention can be administered orally in the form of tablets, pills, or the granulate may be loose filled into capsules. The tablets can be prepared by techniques known in the art and contain a therapeutically effective amounts of the PPI compound and such excipients as are necessary to form the tablet by such techniques. Tablets and pills can additionally be prepared with enteric coatings and buffering systems such as those described above to protect the PPI. The coating may be colored with a pharmaceutically accepted dye. The amount of dye and other excipients in the coating liquid may vary and will not impact the performance of the extended release tablets. The coating liquid generally comprises film forming polymers such as hydroxypropyl cellulose, hydroxypropylmethyl cellulose, cellulose esters or ethers (such as cellulose acetate or ethylcellulose), an acrylic polymer or a mixture of polymers. The coating solution is generally an aqueous solution or an organic solvent further comprising propylene glycol, sorbitan monoleate, sorbic acid, fillers such as titanium dioxide, a pharmaceutically acceptable dye.

One skilled in the art, taking into account above teachings will be readily able to formulate oral dosage forms containing a PPI that is released in accordance with the threshold concentrations, also taught above. Thus, for example, when a controlled or extended release dosage form is employed, the drug should be released such that the plasma concentration threshold is met during the period the drug is released. Hence, when a extended release dosage form is employed according to the present invention, there is a continuous release of the first and second dose of the PPI, and preferably, the plasma level of PPI is maintained above the threshold level during at least part of the combined release of the first and second dose of PPI. Alternatively, when a pulsed release dosage form is employed, the first and second pulses of the drug independently should be sufficient to increase the plasma level concentration above the threshold level.

The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved.

The dosage forms of the present invention are administered and dosed in accordance with sound medical practice, taking into account the clinical condition of the individual patient, the site and method of administration, scheduling of administration, and other factors known to medical practitioners. As further guidance, however, in cases where a pulsed release dosage form is employed, drug loading for each pulse is independently and typically between 5 mg and 300 mg, more typically between 20 mg and 200 mg. In cases where an extended release dosage form is employed, typical drug loading for the combined first and second PPI dose in such a dosage form will be in the range of 50 mg to 1000 mg, and more typically 75 mg to 500 mg.

It has also been discovered, that due to the decreasing absorption in the downstream portions of the gastrointestinal tract, it is preferred to load the dosage forms with the PPI such that the second dose is higher than the first dose of the PPI. Hence, in a pulsed release dosage form the second dose is at least 10% more than the first dose, more preferably, the second dose is at least 50% more than the first dose, even more preferably, the second dose is at least 100% to 200% more than the first dose, and most preferably the second dose is at least 200% to 900% more than the first dose. In continuous release dosage forms of the present invention, the increased second dose is reflected in the total drug loading ranges mentioned above.

In cases where the PPI is delivered in pulses, the period between when the first dose begins to be release and when the second dose begins to be released can be separated by varying amounts of time. Preferably, the onset of release of the doses are separated by between 2 hours and 20 hours, more preferably between 3 hours and 16 hours and most preferably between 4 hours and 12 hours. Of course, any of the pulses released from such a dosage form should achieve the threshold concentration and maintain plasma concentrations above such threshold for at least 30 minutes, preferably one hour to 2 hours, and more preferably between 2 hours to 8 hours. In cases where there is no time separating the pulses, or when the release of the PPI is delivered in an extended or continuous form, the time period for release is also variable but preferably the release is provided over the course of 4 hours to 8 hours, more preferably 3 hours to 12 hours and most preferably 2 hours to 20 hours. Additionally, during this release it is preferable to hold the plasma concentration above a threshold level for at least 4 hours, more preferably at least 6 hours, even more preferably at least 8 hours and most preferably for at least 12 hours.

It will be understood, of course, that dosage forms formulated according to the present invention can be tested empirically in animal and/or human models to determine the appropriate pK parameters resulting from a given dosage form.

Dosage forms of the present invention are exemplified, but not limited to, Examples 1-3. Typically, the gastric emptying time varies between 30 to 90 minutes (when the patient is under fasting conditions). The dosage forms of lansoprazole described in Example 1 were dosed in healthy human subjects. The pharmacokinetic parameters including exposure and their impact on 24-hour gastric pH were determined. An ambulatory pH-monitoring unit, Digitrapper pH100 (Medtronic) was utilized for making the pH measurements. The unit was calibrated before each use, using standard buffers. A disposable, single-channel, antimony pH probe was inserted into the stomach via the flares. Gastric pH was continuously monitored from 10 minutes before to 24 hours after the dose administration. Gastric pH was typically sampled every four to six seconds. While these dosage forms demonstrated differences in the systemic exposure the effect on the gastric pH elevation was comparable.

In a second study, one of the above-mentioned dosage forms (Formulation H) was compared to a commercially available product esomeprazole (Inexium®) with respect to elevating 24-hour gastric pH elevation. Formulation H was superior to Inexium®.

It will be understood here and throughout the text that both Inexium® and Nexium® refer to dosage forms of the PPI esomeprazole, marketed either in Europe or the U.S., respectively.

In a third study, esomeprazole 40 mg (Nexium®) was compared to commercially available lansoprazole 60 mg (2 capsules of Prevacid® 30 mg) with respect to elevating 24-hour gastric pH elevation. The pharmacokinetic parameters including exposure were also determined. Esomeprazole 40 mg was superior to lansoprazole 60 mg in elevating the 24-hour gastric pH on Day 5.

Based on these results, it can be concluded that Formulations A-H provide superior pH control for a period of 24 hours when compared to a 40 mg dose of esomeprazole or a 60 mg dose of lansoprazole. The exposure with these dosage forms is either similar to or slightly higher than that observed with a 60 mg lansoprazole dose. However, this exposure is lower than the exposure observed after administering lansoprazole 60 mg twice a day (total daily dose of 120 mg). Thereupon, these dosage forms enhance the therapeutic efficacy of lansoprazole. These dosage forms are also likely to provide superior night-time acid control and this effect will benefit the patients suffering from nocturnal acid breakthrough.

The following examples are provided to further illustrate the present invention and not intended to limit the invention.

### Example 1

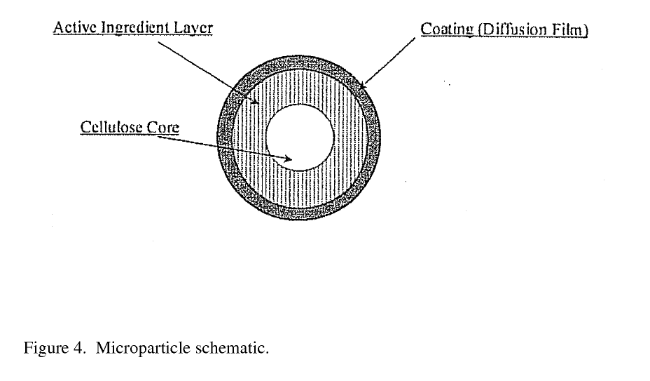

A lansoprazole sustained-release (SR) dosage form (hereinafter “Lansoprazole SR”) was designed to slowly release lansoprazole throughout the upper gastrointestinal (hereinafter “GI”) tract. The tablets contain Lansoprazole SR microparticles coated with a diffusion coating and stabilizers to protect the acid labile drug from degradation when released in the stomach. The Lansoprazole SR microparticles contain an inert microcrystalline cellulose core, an active-ingredient layer and a coating (diffusion film) as shown in the schematic in FIG. 4.

The dissolution profile is the result of lansoprazole diffusion through the diffusion film (pores are formed in the film after ingestion that allow the lansoprazole to diffuse through the diffusion file) and is independent of the dose.

The tablets described above release at least 10% of the lansoprazole within 30-90 minutes after ingestion with the remainder of the drug being released continuously over an at least a 4 hour period.

The compositions of Lansoprazole SR microparticles for a medium release and a fast release formulation are provided in Table 1. The quantity for all of the ingredients is listed in milligrams.

TABLE 1
Composition of Lansoprazole SR microparticles

Quantity (mg)

| Ingredients | Medium Release | Fast Release |
|---|---|---|
| Lansoprazole² | 120 | 120.0 |
| Microcrystalline Cellulose¹ | 99.4 | 149.1 |
| Hydroxypropylcellulose² | 4.5 | 4.5 |
| Magnesium hydroxide² | 18.6 | 18.6 |
| Polysorbate 80² | 6.0 | 6.0 |
| Ethylcellulose³ | 4.2 | 5.0 |
| Povidone³ | 3.3 | 3.9 |
| Ammonio Methacrylate | 4.2 | 5.0 |
| copolymer³ | | |
| Polyoxyl 40 hydrogenated | 0.9 | 1.1 |
| Castor Oil³ | | |
| Castor oil³ | 0.5 | 0.6 |
| Purified Water³ | q.s.⁴ | q.s. |
| Dehydrated alcohol³ | q.s. | q.s. |

¹Part of the Active Ingredient Layer
²Part of the cellulose core
³Part of the Coating (Diffusion Film)
⁴A sufficient quantity.

As will be appreciated by a person skilled in the art, the difference between the medium release dosage form and the fast release dosage form described in Table I above is in the thickness of the diffusion coating.

### Preparation of Lansoprazole SR Microparticles

Microparticles are prepared using a spray-coating technique in bottom spray fluidized bed equipment.

Step 1: The drug-loaded layer (i.e., the active ingredient layer—See Table 1) is deposited by spraying an aqueous suspension of the drug substance onto neutral microcrystalline cellulose cores in order to obtain drug granules.
Step 2: Granules are coated with the diffusion film (See Table 1) by spraying a hydro-alcoholic solution in order to obtain microparticles with modified release characteristics.
Step 3: Microparticles are blended with excipients and stabilizers.

A description of the stabilizer composition utilized for the different dosage forms is provided in Table 2, below.

TABLE 2
Description of Lansoprazole SR Micropump Dosage forms Blended
with Stabilizers

| | | Active | Stabilizer (mg) | |
|---|---|---|---|---|
| Formulation | Drug Release | lansoprazole (mg) | CaCO₃ | MgO |
| A | Medium | 120 | 0 | 0 |
| B | Medium | 120 | 250 | 0 |
| C | Medium | 120 | 500 | 0 |
| D | Medium | 120 | 250 | 250 |
| E | Fast | 120 | 0 | 0 |
| F | Fast | 120 | 250 | 0 |
| G | Fast | 120 | 500 | 0 |
| H | Fast | 120 | 250 | 250 |

Comparison of 24-hour Gastric pH Elevation Effect Between Lansoprazole Medium Release and Fast Release Dosage forms

The objective of this study was to compare the pharmacokinetics and pharmacodynamics of lansoprazole on Day 1 and Day 5 following once daily oral administration of lansoprazole SR Formulation (medium release) taken with either no buffer (Formulation A), 250 mg (Formulation B), or 500 mg calcium carbonate (Formulation C), or a mixture of 250 mg calcium carbonate and 250 mg of magnesium oxide (Formulation D) for five consecutive days. The same study was also conducted with lansoprazole SR Formulation (fast release) taken with either no buffer (Formulation E), 250 mg (Formulation F), or 500 mg calcium carbonate (Formulation G), or a mixture of 250 mg calcium carbonate and 250 mg of magnesium oxide (Formulation H) for five consecutive days.

These were Phase 1, four-period, randomized, open-label, multiple dose, single center, crossover studies in twenty-four subjects. Healthy male or female subjects between 18 and 55 years of age, inclusive, who met all inclusion criteria were studied. In addition to standard Phase 1 selection criteria, all subjects were documented to be H. pylori negative, and free from significant gastrointestinal abnormality prior to enrollment.

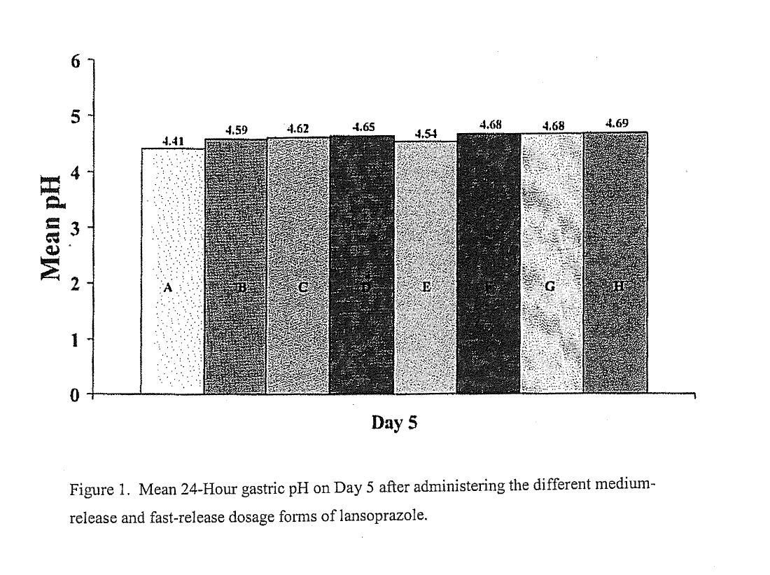

The observed exposure (Area Under Curve determined from the plasma concentration versus time profile) and a comparison of the 24-hour gastric pH elevation on Day 5 for the different dosage forms (Formulations A-H) are presented in Table 3 and FIG. 1.

TABLE 3
Comparison of AUC (exposure) and 24-hour Gastric pH Elevation
on Day 5 of Administration with Different Lansoprazole SR Dosage
forms Blended with Stabilizers

| | AUC | 24-hour Gastric pH |
|---|---|---|
| Formulation | (ng · h/mL) | Elevation |
| A | 4856 | All dosage forms were |
| B | 4982 | comparable |
| C | 4170 | |
| D | 6062 | |
| E | 5054 | |
| F | 5260 | |
| G | 4992 | |
| H | 6465 | |

Conclusion: Although the AUC values observed were variable, the 24-hour gastric pH elevation was comparable for the different dosage forms.

Comparison of 24-hour Gastric pH Elevation Effect Between Dosage Form H and Inexium® (Esomeprazole)

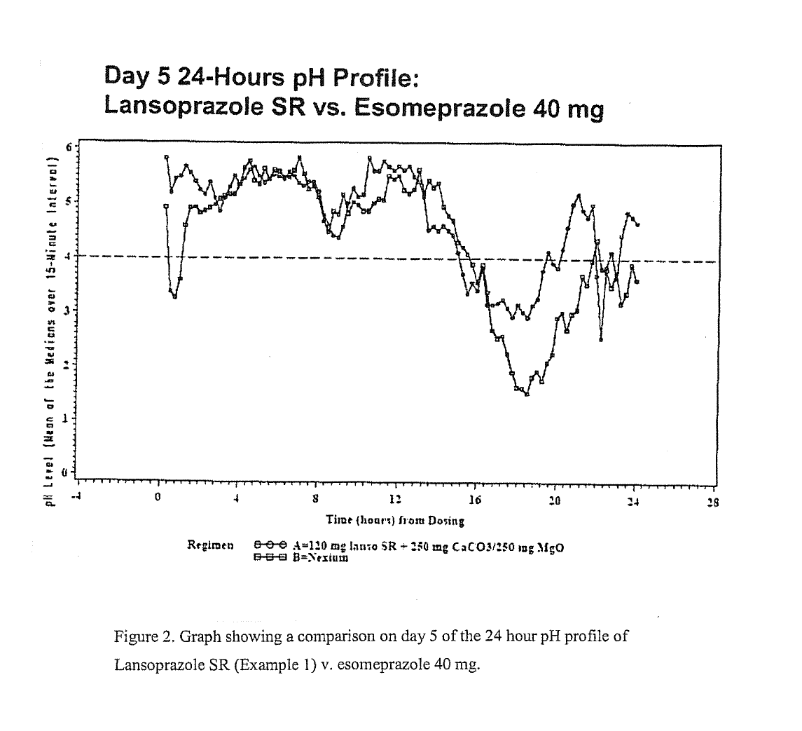

The primary objective of this study was to compare the pharmacodynamics of lansoprazole on Day 1 and Day 5 following once daily oral administration of 120 mg Lansoprazole SR Formulation H taken with 250 mg CaCO₃ and 250 mg MgO to that of 40 mg esomeprazole taken as Inexium® tablets for five consecutive days. Healthy male or female subjects between 18 and 55 years of age, inclusive, who met all inclusion criteria were studied. In addition to standard Phase 1 selection criteria, all subjects were documented to be H. pylori negative and free from significant gastrointestinal abnormality prior to enrollment. This is a 2-period, randomized, open-label, multiple dose, crossover study. Results are presented in FIG. 2.

Conclusion: Lansoprazole Formulation H was superior to Inexium® in elevating 24-hour gastric pH on Day 5. The difference was pronounced in the 16-24 hour pH-monitoring interval indicating that this dosage form would also benefit patients suffering from nocturnal acid breakthrough.
Comparison of 24-hour Gastric pH Elevation Effect Between Prevacid® (Lansoprazole) 60 mg and Nexium® (Esomeprazole) 40 mg

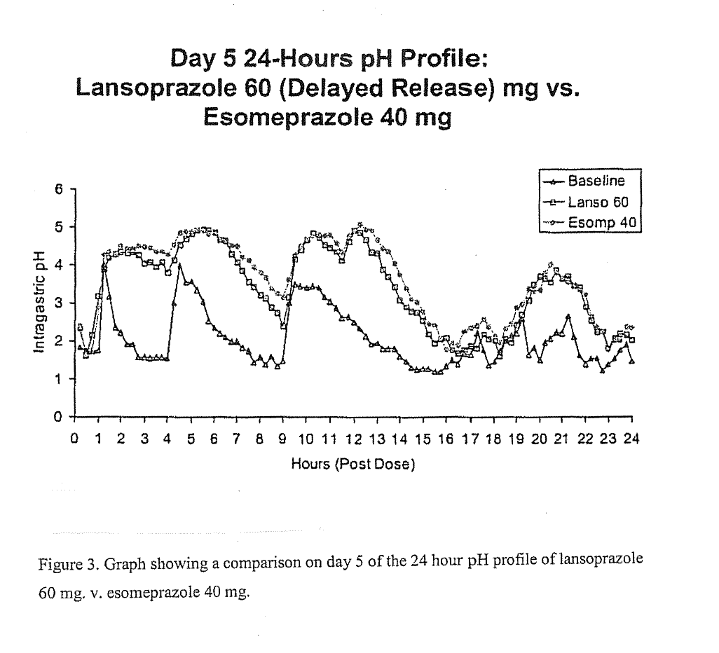

The primary objective of this study was to evaluate the pharmacodynamics of lansoprazole 60 mg once a day (hereinafter referred to as “QD”) and esomeprazole 40 mg QD when administered orally for 5 consecutive days. The pharmacokinetics of lansoprazole and esomeprazole were also evaluated. This was a randomized, open-label, two-way crossover study. Males and females between 18 and 45 years of age were enrolled if they were judged to be in good health based on results of medical history and physical examination, vital signs, ECG, and laboratory tests. The observed exposure (Area Under Curve determined from the plasma concentration versus time profile) and a comparison of the 24-hour pH gastric pH elevation on Day 5 for Prevacid® 60 mg and Nexium® 40 mg are presented in Table 4 and FIG. 3.

TABLE 4
Comparison of AUC (exposure) and 24-hour Gastric pH Elevation
Between Prevacid ® 60 mg and Nexium ® 40 mg
on Day 5 of Administration

| | AUC | |
|---|---|---|
| Formulation | (ng · h/mL) | 24-hour Gastric pH Elevation |
| Prevacid ® 60 mg | 4221 | Nexium ® 40 mg was |
| Nexium ® 40 mg | 4080 | superior to Prevacid ® 60 mg |

### Example 2

Prototype bi-layer tablets were designed to release lansoprazole by an immediate release and an extended release mechanism. Layer 1 was designed to immediately release a blend of drug stabilizer (at least 10%) within 30-90 minutes after ingestion. Layer 2 was formulated to release lansoprazole immediately and for an extended duration of time, specifically, continuously over an at least four (4) hour period after ingestion. The composition of these tablets is provided in Table 5.

TABLE 5
Composition of Bi-layer Tablet Dosage forms

| | | | | Sodium |
|---|---|---|---|---|
| | Calcium | Magnesium | | Starch |
| Components | carbonate | hydroxide | Microcrystalline | Glycolate |
| Layer 1 | (mg) | (mg) | cellulose (mg) | (mg) |
| Formulation P | 250 | 0 | 50 | 50 |
| Formulation Q | 150 | 150 | 50 | 50 |
| Formulation R | 250 | 50 | 50 | 50 |
| Formulation S | 200 | 100 | 50 | 50 |

| | | | Poly (ethylene oxide) |
|---|---|---|---|
| | | | POLYOX ™ |
| Components | Lansoprazole | | 900,000 |
| Layer 2 | (mg) | Lactose (mg) | (mg) |
| Same for | 120 | 200 | 80 |
| Formulations P, | | | |
| Q, R and S | | | |

The tablets were compressed on manual Carver single-punch press (Model No. 3912). The components of Layer 2 were blended together and transferred to the tablet die. The die was gently tapped to settle and level the powder blend. The components of Layer 1 were blended and transferred gently to the die cavity (13 mm). The contents of the die were compressed at 0.9 metric tons for 5 seconds to form the bi-layer tablets. The drug release characteristics were evaluated. More specifically, the tablets having the composition and made as described herein were evaluated for dissolution in a dissolution vessel containing 400 mL of 0.01 N HCl for 30 minutes. Subsequently, 500 ml of sodium phosphate buffer (pH 7.3) was added to the dissolution vessel (to give a total volume of 900 mL). The dissolution was conducted by using a paddle method (USP Apparatus 2) at 75 rpm and 37° C.±0.5° C. Sample aliquots were taken at different time intervals, filtered, and analyzed by high performance liquid chromatography. The results are presented in Table 6.

TABLE 6
Dissolution Profiles of the Bi-layer Tablets

| | % Drug Release | | | | |
|---|---|---|---|---|---|
| Description | 1 Hour | 2 Hour | 3 Hour | 4 Hour | 6 Hour |
| Formulation P | 10.6 | 32.1 | 60.7 | 66.6 | 62.6 |
| Formulation Q | 11.7 | 24.9 | 36.8 | 49.4 | 70.9 |
| Formulation R | 13.0 | 26.3 | 37.8 | 45.9 | 61.6 |
| Formulation S | 11.8 | 26.0 | 38.5 | 47.8 | 68.6 |

Conclusion: The 4 prototype dosage forms released greater than 10% of the drug during the first hour and the release of the remaining drug was extended beyond 6 hours.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

What is claimed is:

1. A continuous release dosage form comprising a PPI which is released from said dosage form in two portions, a first release portion and a second release portion, wherein the first release portion is released into the gastric mucosa whereby the PPI acts directly at the local site of action in said gastric mucosa; and wherein the second release portion is released whereby the remaining PPI provides a sustained plasma concentration.

2. A continuous release dosage form of claim 1 wherein in a fasted state at least 10% of the PPI is released in said first release portion and wherein the remaining PPI is released in said second release portion over a period of at least four hours.

3. A continuous release dosage form of claim 1 or 2 wherein the PPI comprises lansoprazole.

4. A continuous release dosage form of claim 3, wherein the dosage faun comprises a dosage range of from between 60-240 mg. of lansoprazole.

5. A continuous release dosage form of claim 4, wherein the dosage form comprises a dosage range of from between 90-180 mg. of lansoprazole.

6. A continuous release dosage form of claim 5, wherein the dosage from comprises approximately 120 mg. of lansoprazole.

7. A method of treating a gastrointestinal disorder comprising administering to a patient in need thereof a continuous release dosage form comprising a PPI which is released from said dosage form in two portions, a first release portion and a second release portion, wherein the first release portion is released into the gastic mucosa whereby the PPI acts directly at the local site of action in said gastric mucosa; and wherein the second release portion is released whereby the remaining PPI provides a sustained plasma concentration.

8. A method of claim 7, wherein in a fasted state at least 10% of the PPI is released in said first release portion and wherein the remaining PPI is released in said second portion over a period of at least four hours.

9. A method of claim 7 or 8, wherein the PPI comprises lansoprazole.

10. A method of claim 9, wherein the dosage form comprises a dose range of between 60 to 240 mg. of lansoprazole.

11. A method of claim 10, wherein the dosage form comprises a dose range of between 90 to 180 mg. of lansoprazole.

12. A method of claim 11, wherein the dosage form comprises approximately 120 mg. of lansoprazole.

13. A non-enteric coated dosage form for immediate and sustained delivery of at least one PPI to parietal cells in the gastric mucosa in a patient in need of treatment thereof wherein said dosage form comprises at least one PPI and at least one non-enteric coating and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/mL for at least 5 hours after ingestion of said dosage form by said patient.

14. The dosage form of claim 13, wherein the PPI comprises lansoprazole.

15. The dosage form of claim 14, wherein the dosage form comprises from between 60-240 mg. of lansoprazole.

16. The dosage form of claim 15, the dosage form comprises from between 90-180 mg. of lansoprazole.

17. The dosage form of claim 16, wherein the dosage from comprises approximately 120 mg. of lansoprazole.

18. A method of treating a patient in need of treatment thereof, the method comprising the step of administering to a patient in need of treatment thereof a non-enteric coated dosage form comprising at least one PPI and at least one non-enteric coating, and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/mL for at least 5 hours after ingestion of said dosage form by said patient.

19. The method of claim 18, wherein the PPI comprises lansoprazole.

20. The method of claim 19, wherein the dosage form comprises from between 60-240 mg. of lansoprazole.

21. The method of claim 20, the dosage form comprises from between 90-180 mg. of lansoprazole.

22. The method of claim 21, wherein the dosage from comprises approximately 120 mg. of lansoprazole.

23. A non-enteric coated dosage form for immediate and sustained delivery of at least one PPI to parietal cells in the gastric mucosa in a patient in need of treatment thereof wherein said dosage form comprises at least one PPI, at least one stabilizer and at least one non-enteric coating and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/mL for at least 5 hours after ingestion of said dosage form by said patient.

24. The dosage faun of claim 23, wherein the PPI comprises lansoprazole.

25. The dosage form of claim 24, wherein the dosage form comprises from between 60-240 mg. of lansoprazole.

26. The dosage form of claim 25, the dosage form comprises from between 90-180 mg. of lansoprazole.

27. The dosage form of claim 26, wherein the dosage from comprises approximately 120 mg. of lansoprazole.

28. A method of treating a patient in need of treatment thereof, the method comprising the step of administering to a patient in need of treatment thereof a non-enteric coated dosage form comprising at least one PPI, at least one stabilizer and at least one non-enteric coating, and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/mL for at least 5 hours after ingestion of said dosage form by said patient.

29. The method of claim 28, wherein the PPI comprises lansoprazole.

30. The method of claim 29, wherein the dosage form comprises from between 60-240 mg. of lansoprazole.

31. The method of claim 30, the dosage form comprises from between 90-180 mg. of lansoprazole.

32. The method of claim 31, wherein the dosage from comprises approximately 120 mg. of lansoprazole.

33. A non-enteric coated dosage form for immediate and sustained delivery of lansoprazole to parietal cells in the gastric mucosa in a patient in need of treatment thereof wherein said dosage form comprises lansoprazole and at least one non-enteric coating and further wherein said dosage form immediately releases at least ten percent (10%) of said lansoprazole in the gastric mucosa of said patient within thirty to ninety minutes after ingestion of said dosage form by said patient and further wherein said dosage form achieves a plasma concentration of said lansoprazole in an amount greater than 300 ng/mL for at least 5 hours after ingestion of said dosage form by said patient.

34. The method of claim 33, wherein the dosage form comprises from between 60-240 mg. of lansoprazole.

35. The method of claim 34, the dosage form comprises from between 90-180 mg. of lansoprazole.

36. The method of claim 35, wherein the dosage from comprises approximately 120 mg. of lansoprazole.

37. A method of treating a patient in need of treatment thereof, the method comprising the step of administering to a patient in need of treatment thereof a non-enteric coated dosage form comprising lansoprazole and at least one non-enteric coating, and further wherein said dosage form immediately releases at least ten percent (10%) of said lansoprazole in the gastric mucosa of said patient within thirty to ninety minutes after ingestion of said dosage form by said patient and further wherein said dosage form achieves a plasma concentration of said lansoprazole in an amount greater than 300 ng/mL for at least 5 hours after ingestion of said dosage form by said patient.

38. The method of claim 37, wherein the dosage form comprises from between 60-240 mg. of lansoprazole.

39. The method of claim 38, the dosage form comprises from between 90-180 mg. of lansoprazole.

40. The method of claim 39, wherein the dosage from comprises approximately 120 mg. of lansoprazole.

41. A non-enteric coated dosage form for immediate and sustained delivery of at least one PPI to parietal cells in the gastric mucosa in a patient in need of treatment thereof wherein said dosage form comprises at least one PPI and at least one non-enteric coating and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/mL for at least 5 hours after ingestion of said dosage form by said patient and further wherein said dosage form is a dosage form other than a micropump dosage form.

42. The dosage form of claim 41, wherein the PPI comprises lansoprazole.

43. The method of claim 42, wherein the dosage form comprises from between 60-240 mg. of lansoprazole.

44. The method of claim 43, the dosage form comprises from between 90-180 mg. of lansoprazole.

45. The method of claim 44, wherein the dosage from comprises approximately 120 mg. of lansoprazole.

46. A method of treating a patient in need of treatment thereof, the method comprising the step of administering to a patient in need of treatment thereof a non-enteric coated dosage form comprising at least one PPI and at least one non-enteric coating, and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/mL for at least 5 hours after ingestion of said dosage form by said patient and further wherein said dosage form is a dosage form other than a micropump dosage form.

47. The method of claim 46, wherein the PPI comprises lansoprazole.

48. The method of claim 47, wherein the dosage form comprises from between 60-240 mg. of lansoprazole.

49. The method of claim 48, the dosage form comprises from between 90-180 mg. of lansoprazole.

50. The method of claim 49, wherein the dosage from comprises approximately 120 mg. of lansoprazole.

51. A non-enteric coated dosage form for immediate and sustained delivery of at least one PPI to parietal cells in the gastric mucosa in a patient in need of treatment thereof wherein said dosage form comprises at least one PPI, at least one stabilizer and at least one non-enteric coating and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/mL for at least 5 hours after ingestion of said dosage form by said patient and further wherein said dosage form is a dosage form other than a micropump dosage form.

52. The dosage form of claim 51, wherein the PPI comprises lansoprazole.

53. The method of claim 52, wherein the dosage form comprises from between 60-240 mg. of lansoprazole.

54. The method of claim 53, the dosage form comprises from between 90-180 mg. of lansoprazole.

55. The method of claim 54, wherein the dosage from comprises approximately 120 mg. of lansoprazole.

56. A method of treating a patient in need of treatment thereof, the method comprising the step of administering to a patient in need of treatment thereof a non-enteric coated dosage form comprising at least one PPI, at least one stabilizer and at least one non-enteric coating, and further wherein said dosage form immediately releases at least ten percent (10%) of said at least one PPI in the gastric mucosa of said patient within thirty to ninety minutes after ingestion of said dosage form by said patient and further wherein said dosage form achieves a plasma concentration of said at least one PPI in an amount greater than 300 ng/mL for at least 5 hours after ingestion of said dosage form by said patient and further wherein said dosage form is a dosage form other than a micropump dosage form.

57. The method of claim 56, wherein the PPI comprises lansoprazole.

58. The method of claim 57, wherein the dosage form comprises from between 60-240 mg. of lansoprazole.

59. The method of claim 58, the dosage form comprises from between 90-180 mg. of lansoprazole.

60. The method of claim 59, wherein the dosage from comprises approximately 120 mg. of lansoprazole.

61. A method of treating chronic cough in a patient suffering from acid reflux, the method comprising the step of administering to said patient the dosage form of claim 1.

62. A method of treating chronic cough in a patient suffering from acid reflux disease, the method comprising the step of administering to said patient the dosage form of claim 13.

63. A method of treating chronic cough in a patient suffering from acid reflux, the method comprising the step of administering to said patient the dosage form of claim 22.

64. A method of treating chronic cough in a patient suffering from acid reflux, the method comprising the step of administering to said patient the dosage form of claim 33.

65. A method of treating a patient suffering from ulcerative colitis, the method comprising the step of administering to said patient the dosage form of claim 1.

66. A method of treating a patient suffering from ulcerative colitis, the method comprising the step of administering to said patient the dosage form of claim 13.

67. A method of treating a patient suffering from ulcerative colitis, the method comprising the step of administering to said patient the dosage form of claim 22.

68. A method of treating a patient suffering from ulcerative colitis, the method comprising the step of administering to said patient the dosage form of claim 33.
